(19) United States
(12) Patent Application Publication
(10) Pub. No.: US 2007/0048365 A1
(43) Pub. Date: Mar. 1, 2007

(54) Edible coded microsubstrate for pharmaceuticals

(76) Inventor: John J. Rao, Tempe, AZ (US)

Correspondence Address: BOOTH UDALL, PLC, 1423 S. HIGLEY ROAD, SUITE 110, MESA, AZ, 85206, US

(21) Appl. No.: 11/213,214

(22) Filed: Aug. 24, 2005

(51) Int. Cl. A61K 9/48; A61K 9/20; A61K 49/00

(52) U.S. Cl. 424/451; 424/464; 424/10.1

(57) ABSTRACT

A system for verifying product authenticity. A verification system may include: coded microstructures contained within a product. In particular implementations, such as pharmaceuticals, the microstructures are edible and, in some cases, even digestible. Glyph coding may be included to enhance the readability of the microstructures that may have been damaged during manufacture. Microstructures may be incorporated into the product so that they are not visible from the outside of the product to increase the difficulty of counterfeiting the product.

### BACKGROUND

1. Technical Field

This document relates to the authentication of pharmaceuticals, and more particularly to the authentication of pharmaceuticals for consumption and application such as creams, pills, liquids, powders, pastes and the like with a plurality of microscopic markers that are unique to the pharmaceutical product.

2. Background Art

The counterfeiting of products throughout the world is a significant problem that costs legitimate product manufacturers tens of billions of dollars every year. Because of the significant market for counterfeit products, many different attempts have been made to minimize counterfeit products through marking products with unique identifiers. One specific solution includes the inclusion of a plurality of patterned microscopic markers pasted on to a surface of a product as disclosed in U.S. Pat. No. 6,708,618 to Tsai, the disclosure of which relevant to the manufacture of microscopic markers is incorporated herein by reference. A market example of this kind of system is made by DataDot Technology USA in Issaquah, Wash. DataDot Technology USA produces very small “dots” that are about the size of a grain of sand, made from a polyester substrate and suspended in a clear adhesive. The adhesive, and suspended “dots”, may be applied to the surface of property that the user desires to track. More complete information is currently available from DataDot Technology USA's web site at datadotusa.com.

Another known method of marking large products includes a one-dimensional coding system such as a bar code. Bar codes have been around for a very long time. The most common example of marking using a bar code is on food packaging at the grocery store. The one-dimensional bar code can uniquely identify the product, or the type of product by its manufacturer. Some limitations to bar codes is that they cannot be applied to every surface, can be removed from packaging if attached, have no error correction (if there is a smudge the code is unreadable), and generally need a flat surface and high contrast to be successfully read. Two-dimensional coding systems conventionally use black and white contrast and, although they can store more information and have some error correction capabilities, have similar damage, surface and contrast issues as one-dimensional coding systems. Additionally, two-dimensional coding systems generally have sensitive “hot spots.” One- and two-dimensional codes have conventionally worked well on paper or on applications that use labels to stick on wrappings or products.

Another type of identification system that has been used to track products to avoid counterfeiting is a Radio Frequency Identification Device (RFID). Conventionally RFID systems identify products by applying labels with computer chips embedded therein to emit radio signals. Once implemented, a product can be tracked without the need for direct visual sight of the product. RFID tags are, however, much more expensive than other forms of labeling, are electronic devices and, therefore, subject to environmental influences such as thermal, magnetic, electrical or mechanical stresses, are subject to falling off or being made inoperable from the environment, and are often difficult to implement for certain product types.

Yet another known system for marking large products is through a coding system called a glyph. A company called InfoGlyph USA has developed a coding system that does not require high contrast, has no “hot spots”, works well on most any surface including rough and curved surfaces, only requires as little as 10% of the mark for decoding and is, therefore, very robust facing damage, and can have embedded images and logos without reducing readability. Several examples of the types of products that have been marked by InfoGlyph USA with their InfoGlyph technology includes, computer chip surfaces, metals, glass, ceramics, tires, plastics, photographs, and even engine, airplane and car parts. InfoGlyph USA's technology is made under patent rights licensed from Xerox Corporation.

Specifically with relation to pharmaceuticals, the World Health Organization estimates that the booming industry for fake medicines alone is greater than $30 billion per year. To date, the only technologies considered adaptable to pharmaceutical marking to verify authenticity include bar coding on the pharmaceutical product bottle labels, RFID tags placed under the pharmaceutical product bottle labels, and directly marking the surface of pharmaceutical pills with unique identifiers. Methods of marking the surfaces of pharmaceutical pills for later reading to verify origin and authenticity are shown and described in U.S. Pat. No. 5,992,742 to Sullivan et al. (issued Nov. 30, 1999) and U.S. Pat. No. 6,799,725 to Hess et al., the disclosures of which are hereby incorporated herein by reference. The process for labeling individual pills, however, is time intensive, limited in the amount and type of information that can be imprinted to the relatively rough surface of a pill, subject to possible distortion from the pills rubbing together during packing and transportation, and regularly counterfeited.

### SUMMARY

In an aspect, this document features a device, method and system for authenticating pharmaceuticals and other products. The device may include a small substrate marked with one or more codes such as an alphanumeric, one- or two-dimensional code or glyph. That is included within the pharmaceutical or product to be identified. The system for authenticating the item may include an electronic reader or scanner that includes a magnifier, the reader configured to scan the microscopic code from the substrate, decode the information contained in the code, and confirm the authenticity of the code in an associated database, such as an Internet database. The method may include steps for manufacturing pharmaceuticals to include substrates within pharmaceutical pills, creams, lotions, syrups, powders, liquids, and other pharmaceutical formulations.

Implementations may include pharmaceutical products containing identifying substrates within the pharmaceutical material rather than or in addition to merely being on a product bottle label or pill surface. Implementations may also include a laser scribing and a cutting system to create the microscopic code on the substrate and cut it to an appropriate size. For orally administered pharmaceuticals, implementations may include edible substrates. Other particular implementations may include color coded substrates and/or shape coded substrates to indicate visually a category of pharmaceutical products or a pharmaceutical origin.

These and other implementations may have one or more of the following advantages. Pharmaceutical products may be more difficult to counterfeit through implementation of internal coding. Pharmacists and other pharmaceutical manufacturers, distributors and users may more easily verify the authenticity of a particular pharmaceutical product and verify specific information about the pharmaceutical product such as the product name, manufacturer, batch number, batch date, expiration date, dosage, manufacture plant, address and phone number of who to contact with further information about the pharmaceutical, and the like.

These general and specific aspects may be implemented using a system, a method, and/or a computer program, or any combination of systems, methods, and/or computer programs. Additionally, the foregoing and other aspects, features, and advantages will be apparent from the DESCRIPTION and DRAWINGS, and from the CLAIMS.

### BRIEF DESCRIPTION OF THE DRAWINGS

Implementations will hereinafter be described in conjunction with the appended DRAWINGS, where like designations denote like elements, and:

FIGS. 1A-1E are views of substrate examples of various shapes and designs;

FIG. 2 is a representation of coated pharmaceutical tablets showing a portion a tablet crushed;

FIG. 3 is a representation of a sectional view of the coated pharmaceutical of a tablet of FIG. 2 taken along section line 3-3;

FIG. 4 is a representation of pharmaceutical capsules showing the contents of a capsule;

FIG. 5 is a representation of a transparent liquid gel capsule showing the contents of the capsule;

FIG. 6 is a representation of a variety of pharmaceutical products each showing the presence of coded substrates within the pharmaceutical material;

FIGS. 7A-7D are representations of various materials onto which a microcode may be placed;

FIG. 8 is a representation of a system for reading a microcode from a substrate;

FIG. 9 is a representation of the inside of the reader of FIG. 8; and

FIG. 10 is a representation of a laser scribing and cutting system for microcoded substrates.

### DESCRIPTION

1. Overview

Authentication device implementations described here may include edible microcoded substrates mixed into pharmaceutical products. Specific implementations may include pharmaceutical products and in some cases pharmaceutical pills. The various implementations may be manufactured using conventional procedures known to those of ordinary skill in the art as added to and improved upon through the procedures described here.

2. Terminology and Definitions

In describing edible coded microsubstrate implementations, the following terminology will be used in accordance with the definitions and explanations set out below. Notwithstanding, other terminology, definitions, and explanations may be found throughout this document as well.

As used herein, “coded” is a term used in its broadest sense and may refer to any combination of one or more of numbers, letters, symbols and the like that may be read and deciphered by a machine.

As used herein, “edible” is a term used in its broadest sense and may refer to any non-toxic, biocompatible, material that may be swallowed and digested or swallowed and passed through a digestive system. “Edible” items are not limited to items that are consumable by humans.

As used herein, “pharmaceutical” is a term used in its broadest sense and may refer to any preparation that relates to the pharmaceutical industry and may be used in a medical treatment including, but not limited to, prescription and non-prescription drugs, placebo, vitamin and other dietary supplements, homeopathic remedies, plant or animal products, consumable herbs, creams, lotions, liquids, syrups, formulations, powders, pills, solids and all other products that may be sold in or associated with a pharmacy.

As used herein, “pill” is a term used in its broadest sense and may refer to any small pharmaceutical material with a defined solid or semi-solid form that is intended for ingestion. Pills include, without limitation, hard and soft capsules, such as gel capsules, granules and pellets, tablets, coated and non-coated pills, dispersible and chewable tablets, gum-pieces, and all other pills packaged in bulk, bottles, paper, foil or blister packs, or otherwise distributed individually or in groups.

3. Microsubstrates

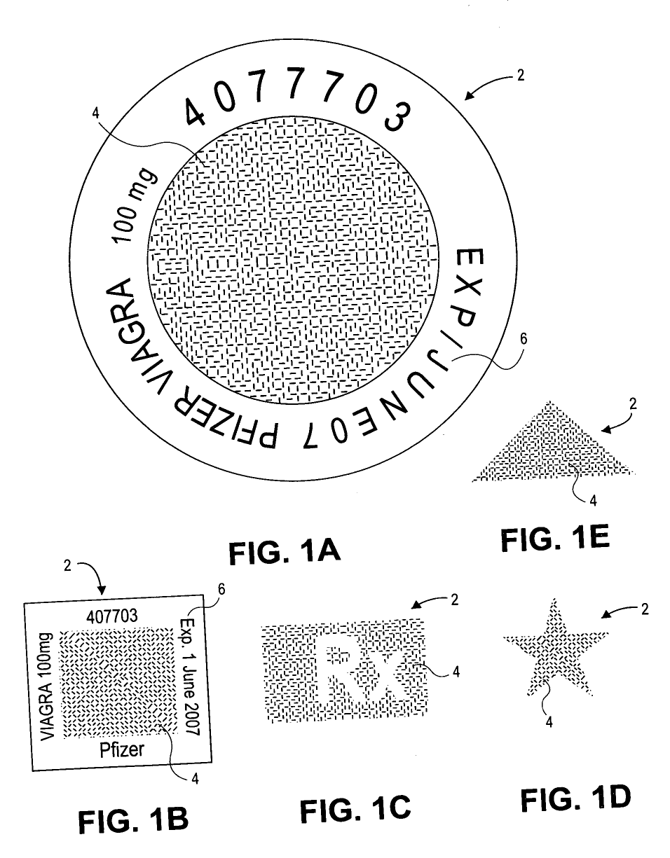

There are a variety of edible coded microsubstrate implementations. Notwithstanding, with reference to FIGS. 1A-10 and for the exemplary purposes of this disclosure, many of the implementations relate to pharmaceutical applications.

Referring to FIGS. 1A-1E, a coded microsubstrate 2 may include substrates in any shape including, but not limited to, a circle, an oval, elliptical, obround, and other circular shapes, a triangle, a rectangle, a square, a pentagon, a hexagon, a star, and other polygonal shapes, as well as any other manufacturable shape. In addition, implementations may include a three-dimensional shape such as a sphere, a cube, a block, a cylinder, a pyramid, and other manufacturable three-dimensional shapes.

Implementations of the microsubstrates 2 may also be color coded, in addition to shape and machine readable coding, to provide particular information about the products into which the microsubstrates are included. For example, in a to pharmaceutical product, a particular shape or color may be used to identify and distinguish particular manufacturer, such as and without limitation, Pfiser, Bristol Meyers Squibb, Haupt Pharma A G, Wagener & Co., SwissCaps, or any other manufacturer, or a particular type of product, such as, and without limitation, for cholesterol, blood pressure, diabetes, weight loss, heart, antibiotic, beauty, and any other pharmaceutical product.

Although it is not required in all implementations, it may be particularly useful to include a two- or three-dimensional code, such as a bar code or a glyph, on the microsubstrate 2. For each of the implementations shown in FIGS. 1A-1E, a portion of the microsubstrate 2 includes a machine readable code portion 4. The machine readable code portion 4 includes information that makes practical sense to a machine, such as a bar code, complex alphanumeric code or glyph. In these particular implementations of FIGS. 1A-1E, the machine readable code portion 4 is a glyph-coded portion, though other codes such as bar codes, dot matrix, alpha-numeric, and other coding is contemplated and feasible as well. For implementations using glyphs and some other coding, like the microsubstrates themselves, the glyph-coded portion 4 of the microsubstrate may be any shape and the shape of the glyph-coded portion 4 need not necessarily correspond with the shape of the microsubstrate 4. Glyphs are used in the implementations shown as examples because of the versatility and robustness of the glyph coding.

Although other glyphs are available and suitable for various implementations of the present invention, a glyph such as the glyph coding generated by InfoGlyph USA, Inc. of Scottsdale, Ariz. (“InfoGlyph”) may be advantageous. InfoGlyph generates a glyph coding that is readable on curved and strongly reflective surfaces, is readable despite significant damage to the glyph-coded area, and is readable despite poor contrast between the marked and unmarked portions of the code. As shown in FIG. 1C, glyph coding may also allow for lettering or logos to be superimposed upon the coded portions. Methods of implementing glyph coding are known in the art and particular methods are shown and described in U.S. Pat. No. 5,449,895 to Hecht et al. (issued Sep. 12, 1995), U.S. Pat. No. 5,521,372 to Hecht et al. (issued May 28, 1996), U.S. Pat. No. 5,091,966 to Bloomberg et al. (issued Feb. 25, 1992), among others, the disclosures of which relevant to the formation and use of glyph coding are hereby incorporated herein by reference.

Coding may include any information useful to identifying the product into which the microsubstrates are included. For pharmaceutical products, it may be desirable to know information such as, without limitation, the manufacturer of the product, the facility at which the product or its parts were manufactured or assembled, the batch number of the product, the product name and strength, dosage information, its manufacture and/or expiration date, its country of origin, and its intended destination or market. By providing an additional verification source for information relating to pharmaceutical products, manufacturers can also provide doctors, pharmacies, boarder searchers, policing agencies, and others concerned with the origin and authenticity of the pharmaceutical a resource for confirming the origin, and therefore the quality and safety, of the product.

In addition to coding that may be read by a machine, such as the glyph coding that may only be practically read by a machine, a human readable code portion 6 may be included in particular implementations. The human readable code portion 6 may include any or all of the information encoded into the machine readable code portion 4. The human readable code portion 6 may be readable by a machine, but it is also readable by and understood by a human.

The microsubstrates used to carry the codes may be made of any material suitable to display the code and allow at least the machine readable portion of it to be read by a machine. The types of materials suitable for various coding types, such as bar code versus glyph versus alphanumeric, will be readily apparent to those of ordinary skill in the art. The size of the microsubstrates may also significantly vary in dimension based upon the anticipated use for the microsubstrate identifier.

In particular implementations, particularly in orally administered pharmaceutical implementations, the microsubstrates may be made edible so that the end user of the pharmaceutical product is not harmed by consuming the product. For edible implementations, although a product that can be digested by the user is not required, it may be advantageous. Edible but nondigestible materials are also useful. Certain plastics, such as MYLAR, ethylene-vinyl alcohol (EVOH), and polyester may be readily eaten and passed through a digestive system without harm to the person or animal that consumed the material. In particular implementations, it is advantageous to formulate the microsubstrate such that it is resistant to the environment within the digestive tract so that the code may be read after it leaves the body with the stool. In such cases, the code may be read after it is separated from the stool, verifying useful information to clinical testing authorities.

The usefulness of this implementation may become particularly apparent when considered in light of the many deaths and illnesses that are blamed upon pharmaceuticals and other edible products. If a doctor or medical examiner can extract more information about what was eaten prior to the death or illness, those potential causes could be ruled out or confirmed. For pharmaceutical companies and doctors, this becomes crucial because through this implementation it can be conclusively determined whether the pharmaceutical taken originated from the company, whether that particular drug was prescribed to that patient, which pharmacy issued the prescription, etc. In a particular implementation, a microsubstrate containing the same code that is included on the microsubstrates within the pharmaceutical products is affixed to an outer surface of a container in which the pharmaceutical products are transferred. At a plurality of check points along the transfer path of the pharmaceuticals, the microsubstrate included on the packaging is scanned and its location and identity verified and stored for later use. For example, and without limitation, the microsubstrate may be scanned within the manufacturing plant, as the pharmaceutical product leaves the manufacturing plant, as the pharmaceutical product arrives at and/or leaves the distribution warehouse, as the pharmaceutical product arrives at the pharmacy, and possibly even as the end user is given the pharmaceutical product.

This process also may assist law enforcement in determining where along the supply and distribution chain particular pharmaceuticals are being diverted to the streets to stop those providing certain pharmaceuticals illegally. Law enforcement officers regularly find pills illegally in the possession of those they apprehend. Being able to quickly track the source of the pills will further assist officers in reducing the damage caused by improper use of pharmaceuticals. An officer who desires to know the origin of a pharmaceutical product can simply locate a microsubstrate and scan the microsubstrate code with a portable reader. Portable readers may be able to store the data for later checking, or check immediately via a wireless connection such as a satellite, cellular, Bluetooth, radio frequency or other wireless or even wired connection. After a processor associated with a reader checks with an associated database, the officer will be able to immediately identify not only the type of drug, its origin and strength, but also which pharmacy, and possibly even which patient, the drug was delivered to.

Companies, such as Filmquest Group of St. Charles Ill. manufacture many types and grades of polyester films suitable for encoding with machine readable code and forming into microsubstrates of appropriate size. Several particular examples of Filmquest Group films that may be suitable for forming microsubstrates for use in implementations include the Questar polyester film Types AI-101, BI-101 and CI-101. Each has a thickness of approximately 100 microns, are non-toxic if swallowed, and, like other polyester films are capable of being marked with a code.

Although the thickness of the microsubstrate is not crucial to particular implementations, it is desirable to make the microsubstrates small so that they are easily eaten and passed. There are printable films as thin as 3 or 4 thousandths thick that are also suitable for particular implementations where thickness is a crucial factor. Also, although the width and height of the microsubstrate is not crucial, it also should be small in edible implementations so as to be easily eaten and passed. The size of the product into which the microsubstrate will be incorporated will be a factor in determining an appropriate size for a particular implementation. Those of ordinary skill in the art will readily be able to select a microsubstrate dimension large enough to print a machine readable code, yet small enough to fit one or more within the product. For pharmaceutical implementations, it is believed that one appropriate size is about 20 to 50 thousandths of an inch. Microsubstrates currently marked with an alpha numeric code used to mark the surface car parts by a company called DataDot Technology USA of Redmond, Wash., has a thickness of about 4 thousandths of an inch thick and about 25 thousandths of an inch wide.

In addition to merely non-toxic microsubstrates, edible microsubstrates of particular implementations may be made of a digestible substance, such as a protein base material. Commonly used digestible substances include, without limitation, protein base materials including gelatin, collagen, hard or soft keratin, and other materials including waxes, polymeric materials (water-based or otherwise), sugar based substrate or certain water-soluble plastics such as polyvinyl alcohol (PVOH).

The use of gelatin as a packaging for pharmaceutical products is known in the industry. Vegetable-based and animal-based versions are also available to distinguish when there is a need for kosher pharmaceutical products and microstructures. A company called ProGel, of the Caldas Department of Western Colombia, manufactures gelatin coatings for pharmaceutical products. Gelatin may be colored, made hard or soft, remain solid in cold water but dissolve in warm water, can be made tasteless and colorless if desired, or can be made to include a taste to minimize the often unpleasant taste of pharmaceutical materials.

The chemistry of gelatin in particular, as well as many other digestible materials, allows it to be manufactured to include particular versatile characteristics useful for using the gelatin in many different environments. Gelatin has good affinity with other colloids such as carragents, agars, alginates, and pectins, and is also compatible with the acids, sugars, polysaccharides and with the colors and flavors normally used in the different formulas. The viscosity of gelatin should be considered when selecting a gelatin for a given use. Gelatin varies from 25 to 60 milipoises at 60iC, measured at 6.66% of concentration, and increases with the concentration and descends with the temperature. The viscosity is also affected by the pH of the solution, due to the physical-chemical changes that occur in the molecule of gelatin at different pH values. In practicality, what this means is that when a microsubstrate is formed of gelatin for mixture with a particular formula for a pharmaceutical or other edible mixture, the gelatin and formula for the mixture may be adapted so that the gelatin microsubstrates are not dissolved and do not otherwise react with the mixture, even a mixture containing liquid, thus preserving the coding placed on the microsubstrate. ProGel products are available through the Santon Trading Corporation of Adelaide, South Australia.

As mentioned earlier, microsubstrates may be formed into any shape. With reference to FIGS. 7A-7C, If a code such as the glyph produced through InfoGlyph, USA (discussed above) is used, not only can the microsubstrates 2 be formed in flat configurations (FIG. 7A), but also curved configurations (FIGS. 7B and 7C). This ability, and incredible advantage for robustness in reading the codes despite deformities, damage and contrast, reduces the need to absolutely protect the microsubstrates during manufacture of the pills and allows for mixture of the microsubstrates with the pharmaceutical material during manufacture of the pill rather than merely printing a code on the pill on a layer after the pill has been formed or adding a label layer to the surface of the pill. As shown in FIG. 7D, and mentioned earlier, use of a glyph code also allows for transparent or virtually/nearly transparent microstructures, or solid colored microstructures, printed with the code in implementations where it is desirable to hide the microsubstrates (such as in a cream, powder, liquid, or even a pill). In such cases, the microsubstrates can appear to simply be part of the pharmaceutical material forming the pharmaceutical product rather than a coded substrate. Through laser scribing the microsubstrates, minute differences in the color and/or texture of the surface of the material of the microsubstrate can be formed and scanned in addition to, or instead of merely inscribing alphanumeric lettering or other codes that require high contrast to read.

3. Pills

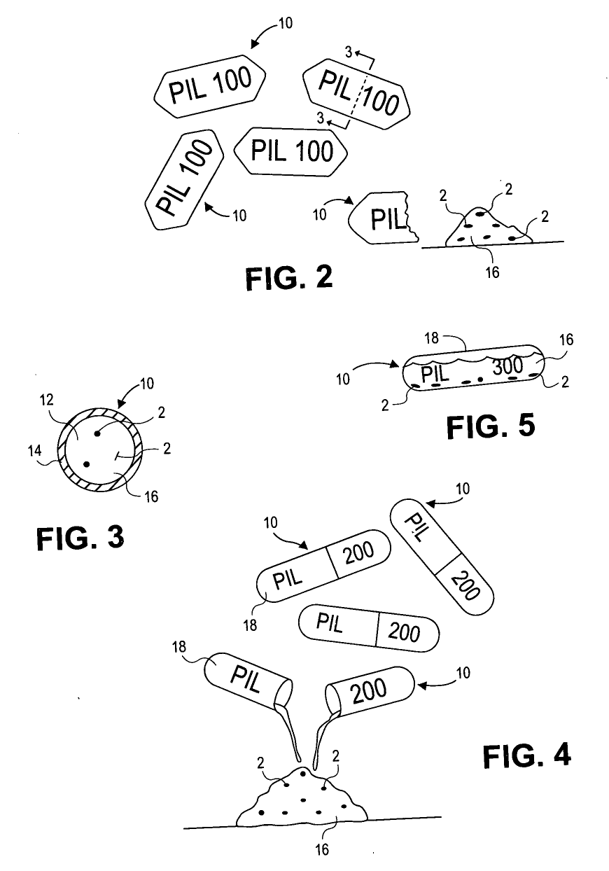

FIG. 2 illustrates a representation of a pill 10 that includes coded microsubstrates 2 within the pill. It is known in the art of pill manufacture to label the outsides of pills with identifying indicia, and even bar codes. Codes made visible and/or readable on the outsides of pills, however, can be difficult to mark, more easily changed and counterfeited than non-visible codes, and when bar codes and alphanumeric codes are used, are more subject to distortion and damage than non-visible codes.

Particular implementations include coded microsubstrates 2 contained within a pharmaceutical core 12 of the pill 10. The pharmaceutical core 12 of the pill is the portion of the pill 10 in which the pharmaceutical material is contained, not merely an outer layer 14 (FIG. 3) of the pill 10. In FIG. 2, a portion of a pill is illustrated in a crushed state to show both the pharmaceutical material 16 and the coded microsubstrates 2. The microsubstrates 2 are not visible from the outside of the pill 10. In particular embodiments, the microsubstrates 2 may be at least partially visible but are not readable from the outside of the pill 10. Throughout this disclosure, non-visible may be used as an example of particular implementations, but non-readable implementations are equally contemplated in each of those examples. Methods of manufacturing pills of many varieties are well known in the art of pill manufacture and one of ordinary skill in the art will readily understand how to incorporate microsubstrates of the various implementations into the pharmaceutical core from this disclosure.

FIG. 3 is a sectional view of a pill 10 of FIG. 2 taken along section lines 3-3. The pill 10 includes a pharmaceutical core 12 comprising pharmaceutical material 16 and coded microsubstrates 2. In addition to pharmaceutical material 16, the pharmaceutical core 12 generally also comprises binder and filler materials and taste masking agents. The outer layer 14 of the pill 10 shown in FIG. 3 was represented in this image to represent not only the outer surface of the pharmaceutical core 12, which is included in the term “outer layer” as it is used herein, but also any protective coatings, enteric coatings, moisture protective coatings, aqueous film coatings, polymer coatings, immediate release coatings, sugar coatings, and any other coatings included around the pharmaceutical core 12.

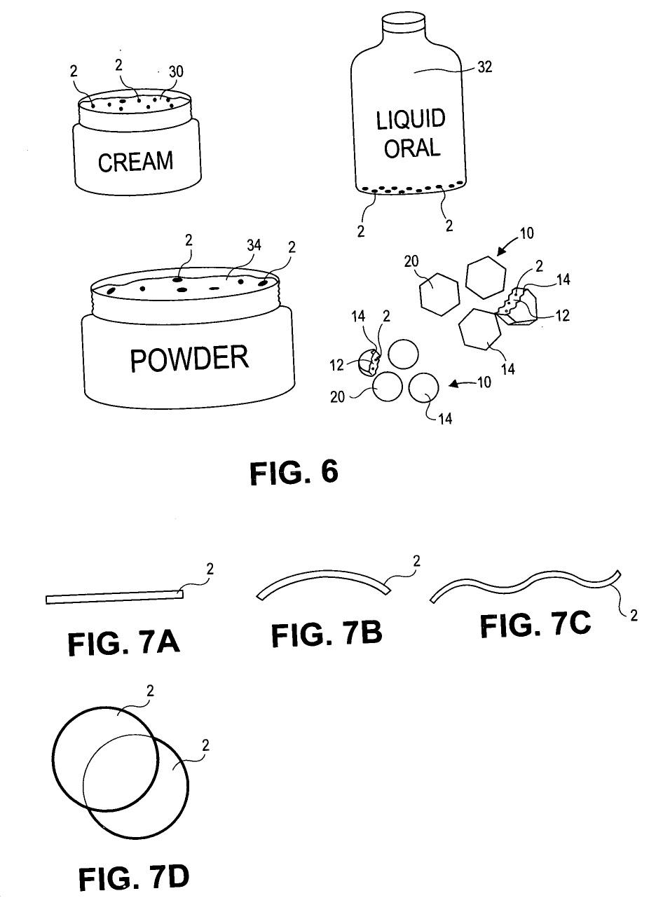

As can be seen in FIGS. 4 and 5, implementations of the invention include pills 10 having powdered, granule or loosely packed pharmaceutical material 16 within a capsule 18 (FIG. 4), and liquid pharmaceutical material 16 within a capsule 18 (FIG. 5). FIG. 6 includes, among other examples of pharmaceutical implementations, dispersible and chewable tablets 20 that do not necessarily include a coating around the pharmaceutical core, but do include an outer layer 14 comprising the outer surface of the pharmaceutical core 12. Note that a majority of the coded microsubstrates 2 are not visible outside of the pharmaceutical core. In particular implementations, the majority of the microsubstrates 2 are not externally visible on the pill.

4. Other Pharmaceutical Implementations

In addition to application in the pill aspect of the pharmaceutical products industry, coded microsubstrates may be included in other pharmaceutical product implementations. For example, as represented in FIG. 6, coded microsubstrates 2 may be incorporated into topical creams 30, such as lotions, beauty creams, antibiotic creams, and any other pharmaceutical creams or topical application. Because, through use of a transparent or virtually transparent microsubstrate and a glyph such as that produced by InfoGlyph USA (discussed previously), a coded substrate may be made virtually transparent, microsubstrates included within ajar or bottle of cream may go completely unnoticed by the user, and may not be visible on the user's skin once applied. Like with the pill examples, the microsubstrates are included within the pharmaceutical material of the cream rather than merely applying the microsubstrate to a surface or otherwise externally visible portion of the material or packaging.

As another example, an implementation includes microsubstrates 2 within a pharmaceutical liquid product 32, such as a cough suppressant or antibiotic. Similarly, the microsubstrates 2 may be included within a powder product 34, such as a dietary supplement or drink mix.

5. Other Non-Pharmaceutical Implementations

In addition to an unlimited number of pharmaceutical implementations, other implementations are also contemplated and possible. These listed here, and many others, will become readily apparent from Applicant's disclosure. For example, other food items may now be labeled simply and unobtrusively to identify its origin and relevant information before and after it has been eaten. Anything edible, therefore, may now be labeled using principles and learned from the implementations disclosed.

Other manufactured goods may be manufactured to include a plurality of microsubstrates therein to later identify their authenticity. For example, sporting equipment (or any other trademarked product), roads, buildings, automobile parts, and the like, may be manufactured to include a plurality of appropriately coded microsubstrates included within the product, and not visible from the outside of the product, that can be used to verify the authenticity or origin of the product if there was ever a need to know its origin with surety.

One particular example of when knowing of a product's origin with surety is significant is not only at the purchase of the product, but if the product failed and caused injury, or failed at all. By allowing the manufacturer to identify that the product is not a counterfeit and that the product originated from a particular manufacturing batch with a known manufacture date and conditions, the manufacturer can better honor warranty claims and avoid future failures. By including microsubstrates within the product manufacture materials and not merely visible from a surface of the product, the products are more difficult to counterfeit.

6. Reading the Microsubstrates

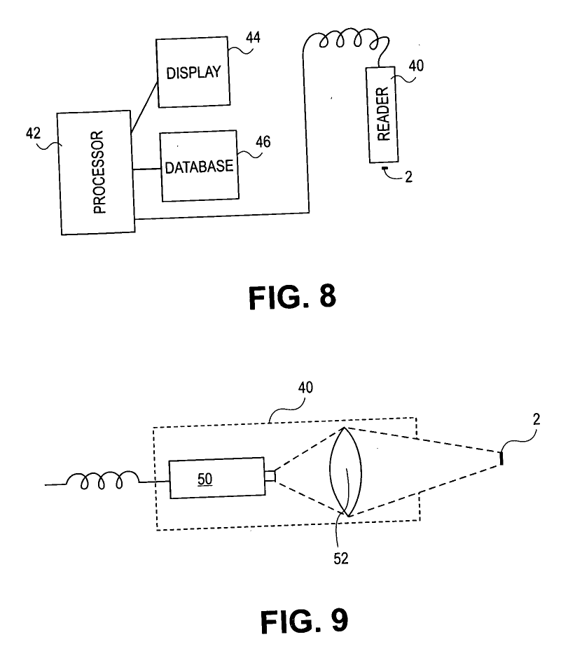

Due to the typically very small size of the microsubstrates in the various implementations, a magnified reader increases the ease of reading the substrates. For the glyph codes, it is believed that the Infoglyph USA data structure has not yet been used on such a small scale previous to the present disclosure. The block diagram of FIG. 8 includes a microcode reader 40 coupled to a processor 42. The processor is configured with programming to enable the processor to receive input from the reader, recognize the data received, decode the data received to identify information about the microsubstrate being read, compare the information with information within a database 46 associated with the processor 42, and display the information on a display 44 associated with the processor. The database 46 may be directly coupled to the processor 42 or may be coupled to the processor 42 through a remote connection such as a wide area network (WAN), local area network (LAN), wireless network, Bluetooth, Internet connection, or any other database access and connection known in the art. Any other components of the system may also be coupled together in the same manner. The processor 42 may be incorporated in whole or in part into the reader 40 or may be maintained as a separate component coupled to the processor. The display 44 may comprise a computer monitor or other monitor common with a processor, whether using display technology as complicated as a plasma display or as simple as a light emitting diode display or anything in between. Alternatively, or additionally, the display 44 may include a printer or other paper-based display onto which the information may be printed. Still again, the display 44 may include a projection-based display to project light to a surface from which the information may be viewed.

As shown in FIG. 9, an implementation of the reader 40 may comprise a digital or analog reader camera 50 comprising electronics and, in some implementations, processors and data storage, and a magnifying lens 52. By magnifying the machine readable coding on the microsubstrate 2 and automatically reading the coding with a reader 50, the code may quickly be decoded and the information reviewed. The reader 40 may be coupled to another support, such as a stand or table, to assist the user in steadying the reader 40 if desired. Alternatively, a hand-held reader may be used. Small readers that may be adapted and programmed by those of ordinary skill in the art to read glyph coded microsubstrates are available from Microscan Systems, Inc. of Renton, Wash.

7. Forming and Marking Microsubstrates

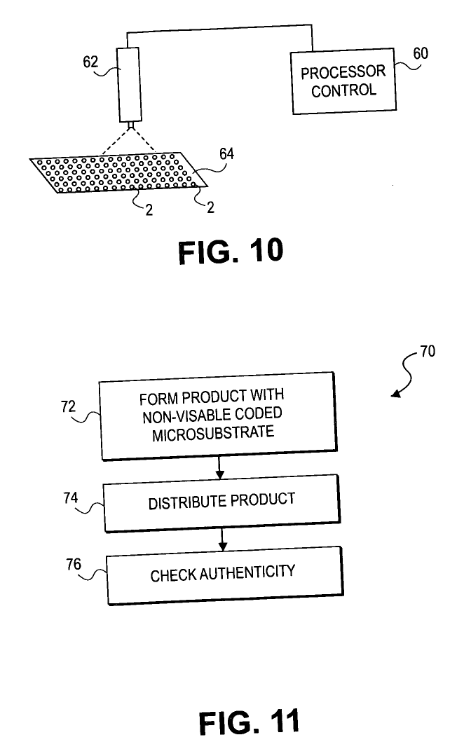

With reference to FIG. 10, there are many different methods by which the microsubstrates may be marked and formed. For example, in one implementation, a processor controller 60 may be used to guide a laser 62 to scribe and cut each of the microsubstrates 2 from a sheet 64 of material. The laser 62 and associated controller 60 may be programmed to modify the intensity of the laser 62 as it passes across the sheet 64 to selectively cut and scribe the sheet 64 to form the microsubstrates 2. Because of the incredibly small size required for the microsubstrate printing, ink printing is not presently possible to achieve high enough resolution for the size of the microsubstrate. Accordingly, laser scribing, or marking, is preferable and is capable of producing the resolution needed to create microsubstrates with marking small enough that even the glyph codes may be displayed clearly through magnification. Laser cutting and scribing systems are available from Laser Cut, Inc. of Branford, Conn., and Trumpf Laser Marking Systems AG of Ausserfeld, Switzerland having the capacity to scribe and cut appropriate coded microsubstrates of many of the implementations disclosed and contemplated herein.

Because the glyph code does not need 100% or even a majority of the code displayed to be decoded, a sheet of appropriate material may be scribed with a code that repeats often enough so that when substrate shapes are cut from the sheet, enough of the code shows in the cut microsubstrate shape to decode the code. Alternatively, the codes may be added at the time the sheets are cut to more accurately include more of the code within the shape boundaries. Those of ordinary skill in the art of laser scribing and cutting will understand and be able to readily apply the most efficient manufacturing techniques to result in microsubstrates with readable codes.

Alternatively, the microsubstrate shapes may be cut from a sheet by stamping, or, in more difficult circumstances, may be injection molded to form the microsubstrates and then laser printed.

8. Use

Implementations are particularly useful in pharmaceuticals. However, implementations are not limited to uses relating to pharmaceuticals. Rather, any description relating to coded microstructures in pharmaceuticals is for the exemplary purposes of this disclosure, and implementations may also be used in a variety of applications with similar results for a variety of products, such as concrete structures with coded microsubstrates mixed into the concrete, individually manufactured products for sale, bulk-manufactured products, and the like.

In describing the use of implementations, with reference to FIG. 11, a method 70 for using the coded microsubstrates is disclosed. As with other examples provided herein, the present implementation example relates to pharmaceuticals but is equally applicable to other products. First, the product is manufactured to include a plurality of coded substrates within portions of the product not visible from the outside (Step 72). For pharmaceuticals, this may be accomplished by forming microsubstrates coded for a particular batch of a particular pharmaceutical pill at a particular factory, pouring a plurality of the microsubstrates into the pharmaceutical material mixture prior to forming the material into pills, forming the material into pills with the microsubstrates inside, and possibly coating the pills with a protective covering.

Because the dosing requirements of pharmaceuticals are so exact, the active ingredients in pharmaceuticals are mixed into the overall mixture extremely uniformly to ensure the dosage for any one pill within the batch is substantially identical to the dosage for every other pill within the batch. Pharmaceutical manufacturers even pride themselves on their general ability to mix the pharmaceutical materials so evenly throughout the filler that even the active ingredient that ends up in the right half of a pharmaceutical pill will be the same amount as that in the left half of the same pill. Accordingly, the addition of coded microsubstrates into the mixture will also result in a highly accurate distribution of microsubstrates throughout the mixture. To ensure that there is at least one microsubstrate in each pill, it is preferable to include enough microsubstrates in the mixture to form pills with two or more microsubstrates per pill.

Once the product is formed (Step 72), it may be packaged and then distributed through its normal distribution channels (Step 74). If the manufacturing company, a potential buyer, or anyone else along the line desires to verify the authenticity of the product for any reason, the product may be opened to locate the coded microspheres. For pharmaceutical pills, opening, or crushing, one or more of the pills is sufficient to locate one or more microspheres. As mentioned earlier, color and/or shape coding the microspheres may assist in identifying the general nature of the pharmaceutical pill and provide an immediate verification of certain information about the pill.

After a coded microsphere is located, the authenticity of the product, or information about the coded microsphere and associated product may be obtained by scanning the microsphere with an appropriately configured reader (Step 76).

Many additional implementations are possible. Further implementations are within the CLAIMS.

1. A pharmaceutical pill comprising:
a pharmaceutical material in pill form having an outer layer surrounding an inner pharmaceutical core; and
an edible microsubstrate marked with a machine readable code representing information about the pharmaceutical pill;
wherein the edible microsubstrate is contained within the inner pharmaceutical core and is not readable from the outside of the pharmaceutical pill.

2. The pharmaceutical pill of claim 1, wherein the pill is a capsule.

3. The pharmaceutical pill of claim 2, wherein the pill is a liquid-filled gel capsule.

4. The pharmaceutical pill of claim 1, wherein the pill is a tablet.

5. The pharmaceutical pill of claim 4, wherein the pill is one of a chewable tablet and a dissolvable tablet.

6. The pharmaceutical pill of claim 1, wherein the machine readable code is a glyph.

7. The pharmaceutical pill of claim 6, wherein the edible microsubstrate comprises a glyph portion and a human readable code portion.

8. The pharmaceutical pill of claim 1, wherein the edible microsubstrate is digestible.

9. The pharmaceutical pill of claim 7, wherein the digestible microsubstrate is formed of a protein base material.

10. The pharmaceutical pill of claim 1, wherein the microsubstrate is not visible from the outside of the pill.

11. The pharmaceutical pill of claim 1, wherein the microsubstrate is contained within the pharmaceutical core.

12. A pharmaceutical product comprising:
a pharmaceutical material stored within a pharmaceutical container, the pharmaceutical material having a material boundary; and
a plurality of microsubstrates each marked with a machine readable code representing information about the pharmaceutical material;
wherein the microsubstrates are contained within the pharmaceutical material boundary.

13. The pharmaceutical product of claim 12, wherein the pharmaceutical product is a pharmaceutical pill, the pharmaceutical material is contained within a pharmaceutical core of the pill and the pharmaceutical material boundary is the boundary of the pharmaceutical core.

14. The pharmaceutical product of claim 12, wherein the pharmaceutical material is a pharmaceutical cream, the material boundary is the boundary of the cream and the microsubstrates are contained within the cream.

15. The pharmaceutical product of claim 12, wherein the pharmaceutical material is in powder or pellet form, the material boundary is the boundary of the powder or pellets as a group, and the microsubstrates are within the boundaries of group.

16. The pharmaceutical product of claim 12, wherein the pharmaceutical material is a liquid, the material boundary is the boundary of the liquid, and the microsubstrates are contained within the liquid.

17. The pharmaceutical product of claim 12, wherein the machine readable code is a glyph.

18. The pharmaceutical product of claim 17, wherein the edible microsubstrate comprises a glyph portion and a human readable code portion.

19. The pharmaceutical product of claim 12, wherein the edible microsubstrate is digestible.

20. The pharmaceutical product of claim 19, wherein the digestible microsubstrate is formed of a protein base material.

21. A method of manufacturing a pharmaceutical product for origin verification, the method comprising:
mixing a plurality of microsubstrates into a pharmaceutical material prior to forming the pharmaceutical material into a pharmaceutical product, each microsubstrate comprising a machine readable code representing information about the pharmaceutical product into which it is to be mixed;
forming the pharmaceutical material into a pharmaceutical product;
distributing the pharmaceutical product;
verifying the origin of the pharmaceutical product by opening the pharmaceutical product, locating at least one of the coded microsubstrates, and scanning the code.

22. The method of claim 21, wherein forming the pharmaceutical material into a product comprises forming the pharmaceutical material into a pill with the microsubstrates mixed therein.

23. The method of claim 21, wherein forming the pharmaceutical material into a product comprises forming the pharmaceutical material into a cream in a bottle with the microsubstrates mixed therein.

24. The method of claim 21, wherein mixing a plurality of microsubstrates comprises mixing a plurality of edible microsubstrates into the pharmaceutical material.

25. The method of claim 21, wherein forming the pharmaceutical product comprises forming the product so that the microstructures are contained within a boundary of the pharmaceutical material.

26. The method of claim 21, wherein the machine readable code comprises a glyph.

27. The method of claim 21, wherein forming the pharmaceutical product comprises forming a pharmaceutical pill so that the microsubstrate is not readable outside a pharmaceutical core of the pill formed by the pharmaceutical material.
